(19) United States
(12) Patent Application Publication
(10) Pub. No.: US 2021/0386417 A1
(43) Pub. Date: Dec. 16, 2021

(54) TOOL FOR SPREADING SOFT TISSUES COVERING THE LUMBAR VERTEBRAE

(71) Applicant: ALLYON, Saint Jean Bonnefonds (FR)

(72) Inventor: Lionel SIMON, Solaize (FR)

(73) Assignee: ALLYON, Saint Jean Bonnefonds (FR)

(21) Appl. No.: 17/278,424

(22) PCT Filed: Sep. 19, 2019
(86) PCT No.: PCT/FR2019/052198

§ 371 Date: Mar. 22, 2021

(30) Foreign Application Priority Data
Sep. 21, 2018 (FR) 18/58595

(51) Int. Cl. A61B 17/02

(52) U.S. Cl. CPC A61B 17/025; A61F 2/4611

(57) ABSTRACT

A tool for spreading soft tissues covering the lumbar vertebrae, including a spreader having a back side which faces the soft tissues, a face side opposite to the back side and two longitudinal edges, the spreader including a rear portion forming a handle, and a front portion having a front end designed to serve as a support on a vertebra which can be pivoted in a main plane about a transverse axis that is perpendicular to the main plane, to apply the back side of the spreader to the soft tissues in order to displace the soft tissues, the front portion including at least one front transverse edge which is extended at the front by a support plate that is substantially parallel to the main plane and provided with an outline for hooking onto the vertebra.

The present invention concerns a tool for retracting soft tissue covering lumbar vertebrae, for lumbar surgical operations.

Certain lumbar surgical operations require a tool for retracting soft tissue, which after an incision of the skin and these tissue, allows bearing on a portion of the vertebra located at the bottom of the incision, then to tilt this tool in order to retract tissue located on one side of this incision, in order to access the vertebra for the operation.

A known type of retracting tool, presented in particular by document WO-A1-20155104507, includes in a main plane a tubular guide portion receiving in the rear portion an operating handle, and in the front portion a fork having two bearing points arranged perpendicular to this main plane, to bear on a lumbar vertebra, in particular on a transverse process of vertebra.

After having incised the soft tissue, the fork is lowered to apply its two points on the vertebra, which only makes it possible to locate the entry point of a tool for drilling a pilot hole, and to make the passage through the soft tissue for a pedicle screw tightening in the pedicle of the vertebra.

This retracting tool allows with a minimally invasive technique to locate and puncture the pedicle entry point to place a screw, but does not allow any other work on the surrounding structures requiring lateral or peripheral displacement of the tissue, for example to achieve a graft on the transverse process or to place an intervertebral cage.

In addition, this retracting tool is in contact with the articular process which can be deformed by osteoarthritis. As a result, its constrained geometry makes it unsuitable for all anatomies, at the risk of imposing a poor path for the drilling tool.

Moreover, by applying the contact fork to the junction of the transverse process and the articular process, the retracting tool then obscures this transverse process which prevents access, for example to deposit a graft.

The object of the present invention is in particular to avoid these drawbacks of the prior art.

It provides for this purpose a tool for retracting soft tissue covering lumbar vertebrae, comprising a retractor having a back side designed to face the soft tissue, a face side opposite said back side and two longitudinal edges, this retractor including a rear portion forming a handle, and a front portion having a front end designed to act as a support on a vertebra in order to perform a pivoting in a principal plane about a transverse axis, perpendicular to this main plane, to apply the back side of the retractor on the soft tissue in order to push them back, this tool being remarkable in that the front portion comprises at least one front transverse edge extended forward by a support plate substantially parallel to the main plane and equipped with a hooking contour on the vertebra.

An advantage of this retracting tool is that the support plate constituting a plane parallel to the main plane, occupies in the transverse direction a reduced space equal to the thickness of this plane, and is preferably disposed in the extension of a longitudinal edge of the retractor. This clears the space in front of the retractor, allowing surgical operations to be performed. A support surface is also provided on the outer face of the plate to guide an arthrectomy or insertion of an intervertebral cage.

The retracting tool according to the invention may further include one or more of the following features, which may be combined with one another.

Advantageously, the hooking contour of the support plate is oriented towards the face side. In addition, this hooking contour may include hooking teeth on the vertebra. A safer hooking of the tool is achieved.

Advantageously, the support plate includes rearwards a projecting stop turned towards the face side. This stop, coming on the vertebra, limits the descent of the tool into the patient.

Advantageously, the retractor has in its cross section a convex shape turned towards the back side.

Advantageously, the retracting tool includes a longitudinal distance between the front end of the support plate and the front transverse end of the retractor, which is greater than six millimeters. In this way, a space is cleared in front of the retractor, to carry out operations.

According to an embodiment, the retracting tool includes two substantially parallel support plates, disposed symmetrically on the longitudinal edges of the retractor.

In this case, the tool may include a bridge connecting the two support plates.

According to another embodiment, the retracting tool includes two retractors disposed transversely on each side of the support plate.

Advantageously, the retractor includes radiological marks.

In particular, the tool may include a longitudinal line forming a radiological mark.

In addition, the retracting tool may include a mark having an asymmetry with respect to the median plane of the retractor.

The invention will be better understood and other features and advantages will emerge more clearly on reading the description below given by way of example, with reference to the accompanying drawings in which:

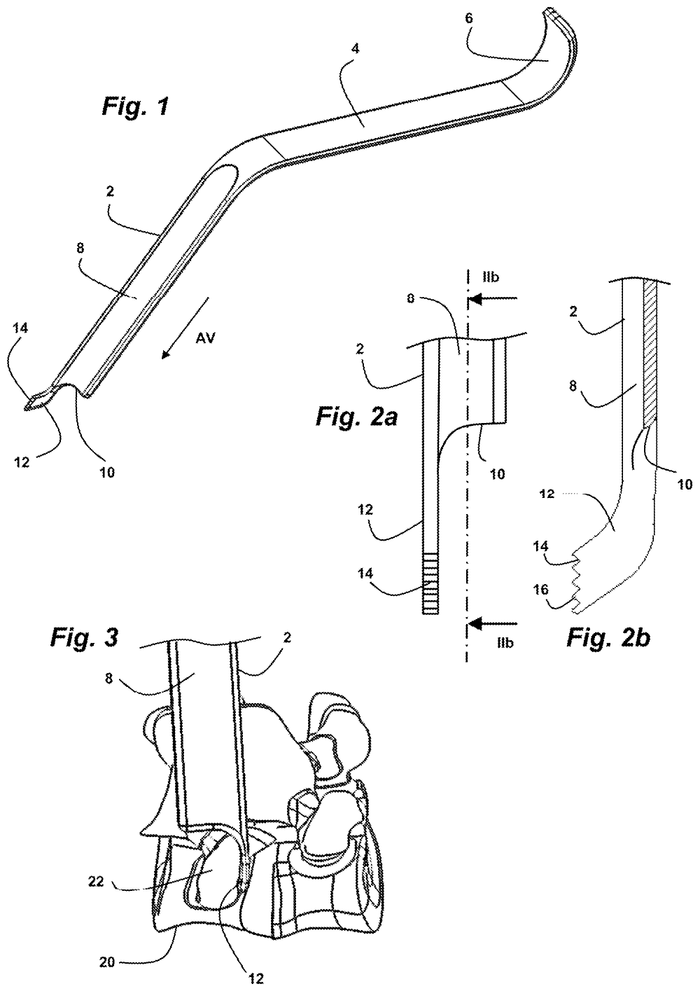

FIG. 1 shows a retracting tool according to the invention;

FIGS. 2a and 2b show the front of the tool, respectively in front view and in section along the median section plane IIb-IIb;

FIGS. 3 and 4 show the placement of this tool on an articular process and bearing against the lateral face of the transverse process;

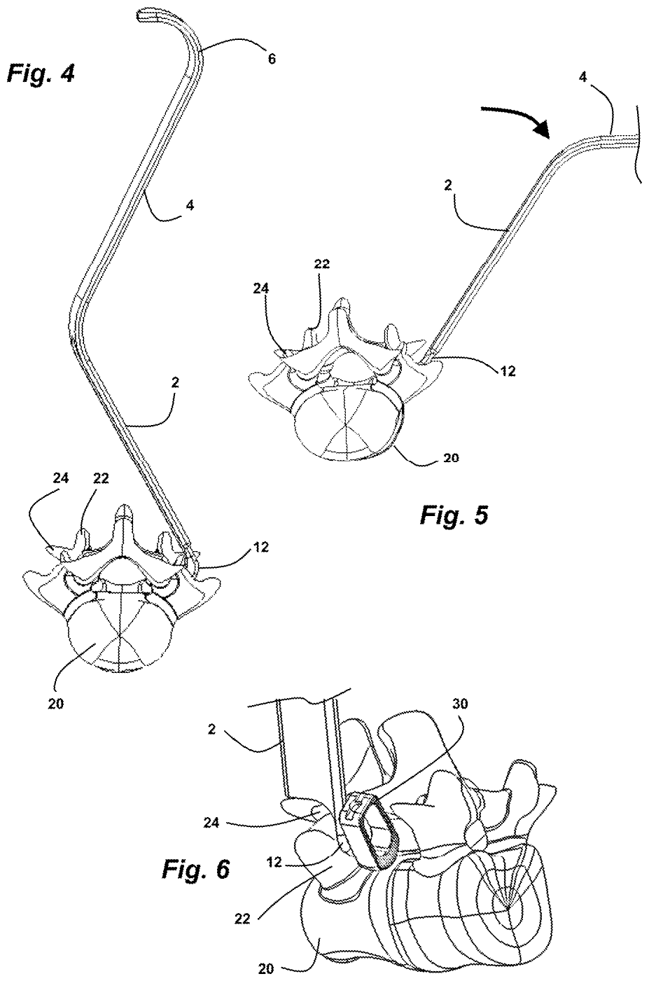

FIG. 5 shows the tilting of this tool to retract the soft tissue located on the back of the retractor;

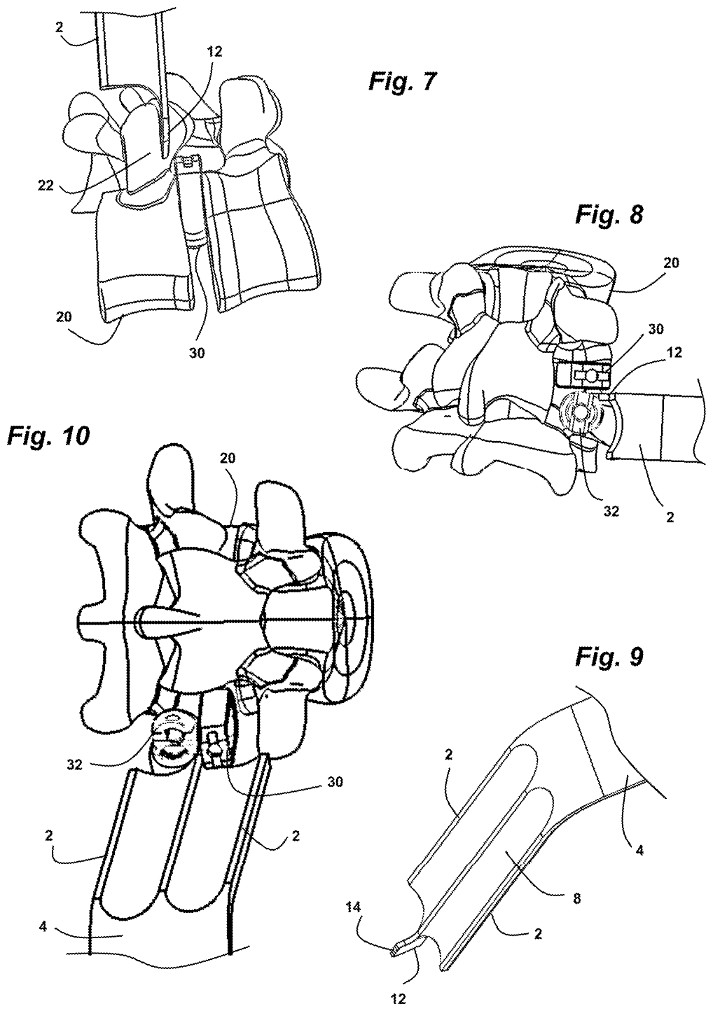

FIGS. 6 and 7 show the insertion of a cage in the intervertebral space after the tilting of this tool, FIG. 7 showing the portion of articular process projecting on the path of the cage, which can ideally be resected by guiding the retractor plate to free this path;

FIG. 8 shows the implantation of a screw next to the cage;

FIGS. 9 and 10 alternatively show a retracting tool with double retractors symmetrical with respect to the support plate;

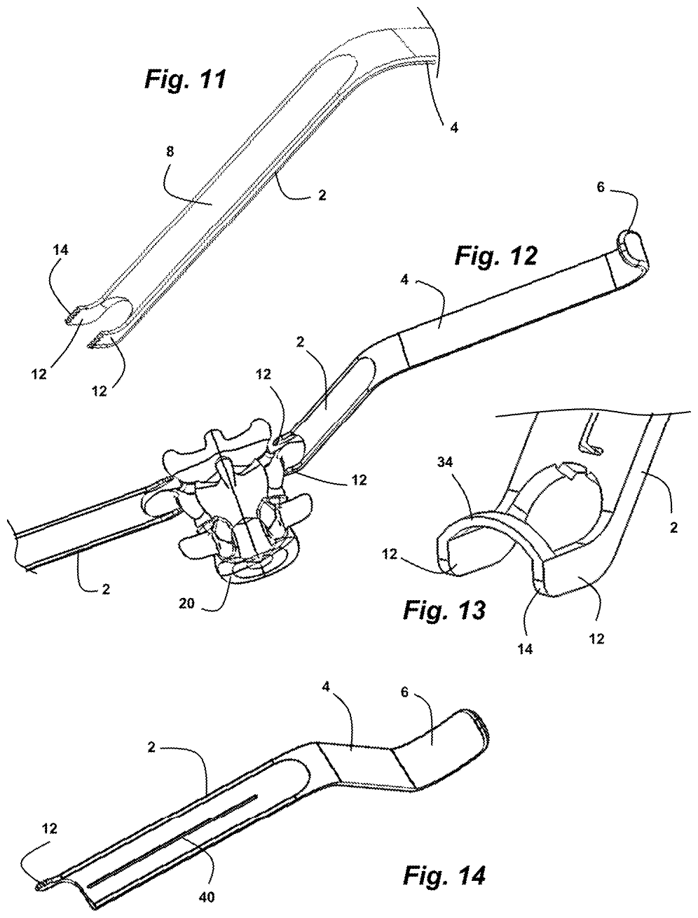

FIGS. 11 and 12 alternatively show a retracting tool with double support plates each disposed in the extension of one of the two longitudinal edges of the retractor, on either side of the front transverse edge;

FIG. 13 shows this tool comprising a bridge connecting the two support plates;

FIG. 14 alternatively shows a retracting tool provided with a visual and radiological mark;

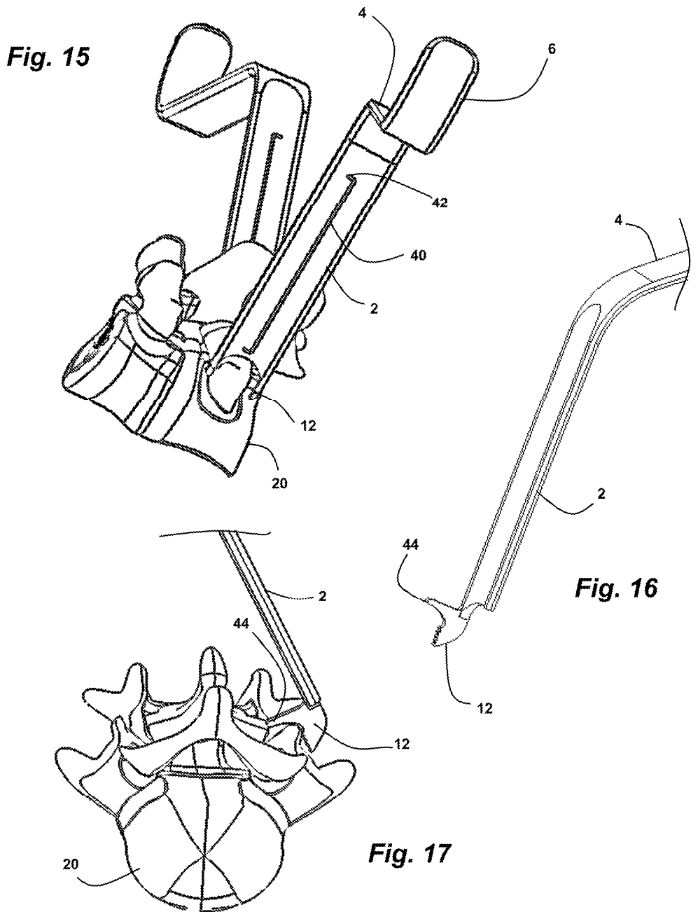

FIG. 15 alternatively shows two retracting tools provided with a visual and radiological mark allowing differentiation of these tools on lateral radiographs; and

FIGS. 16 and 17 alternatively show a retracting tool comprising a support plate equipped with a stop.

FIGS. 1, 2a and 2b show a retracting tool formed in this example in a strip of constant width, cut from a metal sheet and shaped with a curvature disposed in a main plane, comprising a retractor 2 having successively, by starting from the front indicated by the arrow AV, a rectilinear front portion elongated in a longitudinal direction and equipped with a flat support plate 12, then a rear portion defining a rectilinear handle 4 which ends in a rear hook 6.

The handle 4 having in this example a flat cross section, forms with the retractor 2 a fold facing a side called back, having an angle of about 135°. The rear hook 6 facing the face side, opposite the back side, has a regular curvature at an angle of about 120°.

The retractor 2 includes in its cross section a convex shape which is turned towards the back, forming on the face a hollow 8, symmetrical with respect to the median plane of the tool, receiving on each edge a flat portion of reduced width. The front end of the retractor 2 includes a front cross edge 10, which after bending forward is extended by the support plate 12.

The support plate 12, formed by the sheet forming a plane substantially parallel to the main plane, extends the retractor 2 forwardly on its longitudinal edge so as to remain substantially in the width of the strip. The support plate 12 rises towards the face side, to end with a hooking contour 14 arranged in the longitudinal direction, comprising five points 16 distributed over this hooking contour 14.

FIGS. 3 and 4 show after the median incision of the skin and the disinsertion of the muscles of the posterior blade, the descent of the retracting tool on a vertebra 20, then the positioning of the hooking contour 14 to the end of the support plate 12 on an articular process 24 and in apposition on the lateral face of the transverse process 22, this plate being perpendicular to the axis of the vertebral column.

It will be noted that the section of the retractor 2 with its support plate 12 has a reduced thickness, which makes it possible to make a minimum incision on the skin and the tissue for its introduction. A restricted surgery is performed which is minimally intrusive, close to the size of minimally invasive incisions made with the retracting tool according to the prior art presented above. In particular, its geometric configuration provides a deep and precise spacing improving exposure compared to surgical habits using in particular a Beckmann retractor, thus creating a surgery whose invasiveness is intermediate between open surgery and the minimally invasive surgery presented in prior art above.

The rear hook 6 of the handle 4 allows the hand of the surgeon to be wedged or the hooking of a stretched link to maintain the inclination of the retractor 2, or alternatively the hooking of a frame maintaining the distance between two retractors disposed symmetrically. In addition, the hook 6 can be centered on the median plane, or offset laterally to compensate for an offset of the forces given by the support plate 12 arranged along the longitudinal edge.

FIG. 5 shows, after the points 16 of the hooking contour 14 have been bearing against the articular process 24 so as to securely hook the support plate 12, a tilting towards the back of the retracting tool. The convex shape on the back of the retractor 2 pushes back soft tissue without injuring it, so as to clear a workspace towards the median plane of the patient back.

The elongated shape of the retracting tool, comprising the handle 4 and its rear hook 6 facilitating the grip of the tool and giving the surgeon a mark on this handle 4, constitutes an important lever arm making it possible to apply a sufficiently high pressure on the tissue to separate them, while keeping the points 16 bearing against the vertebra 20 in order to prevent it from slipping.

In particular, with the tool according to the invention, several geometries of interest are obtained. The support plate 12 can be applied against the lateral face of the articular process 24, this face serving as a pivot of the tool to cause the separation. The support plate 12 can also be applied against the cranial face of the transverse process associated with the pedicle to be protected in the event of arthrectomy, comprising the cutting of the articular process making it possible in particular to clear a space in order to pass an intervertebral cage.

In addition, the support plate 12 being in the extension of the longitudinal edge of the retractor 2, and the front transverse edge 10 of the retractor 2 being in the longitudinal direction away from the support plate 12, a clearance between this retractor 2 and the transverse process 22 is obtained. This clearance prevents early contact with the posterior face of the transverse process 22 to allow its exposure, in order to deposit the graft if necessary, and to avoid a fracture of this transverse process by the pivoting movement.

Precise access is also obtained facing this retractor in front of its front transverse face 10 which clears all the structures of interest for vertebral surgery, namely the posterior blade, the pedicle entry point, the articular process, the posterior face of the transverse process and, on the side, the intervertebral disc space.

Advantageously, the longitudinal distance between the front end of the support plate 12 and the front transverse end 10 of the retractor 2 is greater than six millimeters, namely at least the radius of a screw to be implanted, and can reach twenty millimeters.

This gives access to the posterior blade useful in the case of a narrow lumbar canal, an access to the pedicle path necessary to place screws, an access to the intervertebral space for the placement of an intervertebral cage, and an access to the posterior surface of the transverse process for the posterolateral graft.

The retracting tool formed from a single piece made of an unalterable material, not comprising moving portions such as a joint or a rack, is indestructible. It can be used alternately from one side to the other, allowing a more precise, in-depth access, with a stability that completely frees up the workspace.

FIGS. 6 and 7 show the outer side of the support plate 12 forming a direction of the cut line of the articular process 24, then allowing the penetration of an intervertebral cage 30 in this cutout, along the support plate.

FIG. 8 shows the placement of a screw 32 on the side of the support plate 12 opposite to the cage 30, this support plate 12 forming a separation between the two implant spaces.

FIGS. 9 and 10 show a retracting tool comprising two parallel retractors 2 each comprising a convex shape, disposed symmetrically with respect to the support plate 12 located between these two retractors 2.

The two parallel retractors 2 allow, from a central support of the support plate 12, to retract the soft tissue located on each side of this support plate 12. It is thus possible for example to simultaneously access to a cage 30 and to a screw 32, by placing the support plate 12 between these two elements.

FIGS. 11 and 12 show a retracting tool comprising a single retractor 2, including two parallel support plates 12 disposed symmetrically on the longitudinal edges of this retractor.

This retracting tool being symmetrical, the same tool can be used on the left and on the right as shown in FIG. 12. In particular, the transverse distance between the two support plates 12 must be sufficient to allow operations between them. Advantageously, a distance comprised between five and twenty-five millimeters is provided.

FIG. 13 alternatively shows a retracting tool with two parallel support plates 12, connected at their front ends by a narrow junction forming a convex bridge 34 turned towards the face side, which strongly stiffens these support plates 12.

FIG. 14 shows a retracting tool including a longitudinal line 40 forming a visual and radiological mark, marked in the hollow 8 of the retractor 2 along its axis of symmetry. In particular, the visual marker can be positioned at such a distance from the support plate 12 that it extends the axis of the vertebral pedicle when this support plate is affixed against the lateral face of the transverse process 22, whose shape is almost invariable from one patient to another, namely a distance of about 9 mm.

The longitudinal line 40 constitutes a mark extending the pedicle path envisaged a priori by the surgeon, it allows a double visual and radiological identification. It is thus possible to correct the final trajectory of the drilling tool with respect to the trajectory indicated by the mark, after visualization of the retracting tool in position on the bone structure of the patient by radiological images during the operation. This ensures optimal placement of the pedicle screw.

FIG. 15 shows a retracting tool comprising at each end of the longitudinal line 40 forming the mark, a small transverse line 42, these two small lines turned transversely on the same side with respect to the pedicle median plane, forming a non-symmetrical mark.

When using two opposing retracting tools as shown in FIG. 15, a symmetry effect is obtained on these two tools with transverse lines 42 turned from two opposite sides. This reversal of direction of these transverse lines 42 forms a marking differentiating the two used retracting tools, giving a visual mark on a radiography allowing each tool to be identified without error.

In particular, the longitudinal line 40 and the perpendicular lines 42 can be produced by a slit in a material opaque to radiological radiation.

FIGS. 16 and 17 show a retracting tool comprising at the rear of the support plate 12, a nose forming a projecting stop 44 remaining in the plane of this plate, which points towards the face side, perpendicular to the longitudinal axis of the retractor 2.

The projecting stop 44 constitutes, during a tilting movement of the retracting tool, a support preventing this tool from plunging too deeply below the level of the transverse.

In general, the retracting tool can be made with any metallic, plastic or other materials which are radio-transparent or radio-opaque. These tools can be disposable, or reusable after sterilization.

1. A tool for retracting soft tissue covering lumbar vertebrae, including a retractor having a back side designed to face the soft tissue, a face side opposite said back side and two longitudinal edges, this retractor including a rear portion forming a handle, and a front portion having a front end designed to act as a support on a vertebra in order to perform a pivoting in a main plane about a transverse axis, perpendicular to this main plane, to apply the back side of the retractor on the soft tissue in order to push them back, wherein the front portion comprises at least one front transverse edge extended forward by a support plate substantially parallel to the main plane and equipped with a hooking contour on the vertebra.

2. The retracting tool according to claim 1, wherein the hooking contour of the support plate is oriented towards the face side.

3. The retracting tool according to claim 1, wherein the hooking contour of the support plate includes hooking teeth on the vertebra.

4. The retracting tool according to claim 1, wherein the support plate includes towards the rear a projecting stop facing the face side.

5. The retracting tool according to claim 1, wherein the retractor has in its cross section a convex shape facing the back side.

6. The retracting tool according to claim 1, wherein the retracting tool includes a longitudinal distance between the front end of the support plate and the front transverse end of the retractor, which is greater than six millimeters.

7. The retracting tool according to claim 1, wherein the retracting tool includes two support plates substantially parallel, disposed symmetrically on the longitudinal edges of the retractor.

8. The retracting tool according to claim 7 wherein the retracting tool includes a bridge connecting the two support plates.

9. The retracting tool according to claim 1, wherein the retracting tool includes two retractors disposed transversely on each side of the support plate.

10. The retracting tool according to claim 1, wherein the retractor includes radiological marks.

11. The retracting tool according to claim 10, wherein the retracting tool includes a longitudinal line forming a radiological mark.

12. The retracting tool according to claim 10, wherein the retracting tool includes a mark having an asymmetry relative to the median plane of the retractor.
